(19) United States
(12) Patent Application Publication
(10) Pub. No.: US 2020/0179402 A1
(43) Pub. Date: Jun. 11, 2020

(54) MONOHYDROXYLATED 17ALPHA-HYDROXYPROGESTERONE CAPROATE FOR REDUCING CONTRACTILITY

(71) Applicant: Indiana University Research and Technology Corporation, Indianapolis, IN (US)

(72) Inventor: Avinash Shivaputrappa Patil, Zionsville, IN (US)

(21) Appl. No.: 16/321,965

(22) PCT Filed: Aug. 3, 2017
(86) PCT No.: PCT/US2017/045265

§ 371 Date: Jan. 30, 2019

Related U.S. Application Data
(60) Provisional application No. 62/370,448, filed on Aug. 3, 2016.

(51) Int. Cl. A61K 31/57; A61K 9/00; A61K 47/44

(52) U.S. Cl. CPC A61K 31/57; C07K 16/44; A61K 47/44; A61K 9/0019

(57) ABSTRACT

A monohydroxylated 17α-hydroxyprogesterone caproate (HPC—OH), 17α-hydroxyprogesteryl 6-hydroxycaproate, is disclosed herein for reducing uterine contractions, reducing inflammation related to contractility, and preventing preterm birth. Additionally, the use of HPC—OH as a diagnostic is also disclosed.

### CROSS-REFERENCE TO RELATED APPLICATIONS

This application claims priority to U.S. Provisional Application No. 62/370,448 filed on Aug. 3, 2016, which is hereby incorporated by reference in its entirety.

### BACKGROUND OF THE DISCLOSURE

The present disclosure relates generally to the use of monohydroxylated 17α-hydroxyprogesterone caproate (HPC—OH) for reducing contractility (both contractile force and contractile frequency), reducing inflammation, and preventing preterm birth. More particularly, it has been found that monohydroxylated 17α-hydroxyprogesterone caproate (HPC—OH), and particularly, 17α-hydroxyprogesteryl 6-hydroxycaproate, more potently reduces uterine contractions as compared to its parent compound, 17-hydoxyprogesterone caproate (HPC).

Preterm birth is a major public health problem, leading to lifelong morbidities in premature newborns and high expenditures for health care systems and insurance companies. Each year, an estimated 15 million babies are born preterm (before 37 weeks gestation) according to the World Health Organization. Globally, preterm birth is the leading cause of newborn deaths (babies in the first four weeks of life) and the second leading cause of death in children under five years. Complications arising from preterm birth include acute respiratory, gastrointestinal, immunologic, central nervous system, hearing, and vision problems, as well as longer-term motor, cognitive, visual, hearing, behavioral, social-emotional, health, and growth problems. Many survivors face a lifetime of disability, including learning disabilities and visual and hearing problems.

If a pregnant woman is determined to be at risk for preterm birth, health care providers can implement various clinical strategies that may include surgical procedures such as cervical cerclage and cervical pessaries, preventive medications, restrictions on sexual activity and/or other physical activities, and alterations of treatments for chronic conditions that increase the risk of preterm labor.

The benefits of 17α-hydroxyprogesterone caproate (I7-HPC) in preventing recurrent preterm delivery in women with history of prior spontaneous preterm birth have been demonstrated in clinical trials (33% risk reduction). These findings renewed the interest of clinical and basic scientists in investigating the role of natural and synthetic progestins in preventing preterm delivery, and further, have led to a search for more effective therapies.

Based on the foregoing, there is a need for an additional or alternative therapeutic for preventing preterm birth with greater efficacy than existing therapies and/or has efficacy for other risk factors associated with preterm birth. It would be further advantageous if the therapeutic was as potent or more potent in preventing preterm contractions and labor as compared to existing medications.

### BRIEF DESCRIPTION OF THE DISCLOSURE

The present disclosure is generally related to the use of monohydroxylated 17α-hydroxyprogesterone caproate (HPC—OH) for reducing the incidence of preterm birth. More particularly, the present disclosure is related to the use of 17α-hydroxyprogesteryl 6-hydroxycaproate for reducing uterine contractions, reducing inflammation related to contractility, and/or preterm birth.

In one embodiment, the present disclosure is directed to a method of reducing the incidence of preterm delivery in a pregnant female subject, the method comprising administering 17α-hydroxyprogesteryl 6-hydroxycaproate to the subject.

In another embodiment, the present disclosure is directed to a method of reducing contractility in a pregnant female subject, the method comprising administering 17α-hydroxyprogesteryl 6-hydroxycaproate to the subject.

In yet another embodiment, the present disclosure is directed to a method of reducing inflammation in a pregnant female subject, the method comprising administering 17α-hydroxyprogesteryl 6-hydroxycaproate to the subject.

In accordance with the present disclosure, methods have been discovered that surprisingly allow for greater potency in reducing uterine contractions and preterm labor. Significantly, the methods of the present disclosure provide treatment to a pregnant female who is susceptible to preterm delivery with monohydroxylated 17α-hydroxyprogesterone caproate (HPC—OH), and particularly, 17α-hydroxyprogesteryl 6-hydroxycaproate, to decrease contractility and inflammation related to contractility with greater potency and effectiveness than the parent compound, 17-hydoxyprogesterone caproate (HPC). Further, 17α-hydroxyprogesteryl 6-hydroxycaproate has been found to have a longer half-life as compared to HPC.

In further embodiments of the present disclosure, it has been found that monohydroxylated 17α-hydroxyprogesterone caproate (HPC—OH) can be used as a diagnostic marker of 17-hydoxyprogesterone caproate (HPC) treatment efficacy. Particularly, by detecting a concentration of 17α-hydroxyprogesteryl 6-hydroxycaproate in a subject, 17-hydoxyprogesterone caproate (HPC) treatment can be adjusted to provide more effective treatment in a subject.

Accordingly, in one further embodiment, the present disclosure is directed to a method of monitoring the efficacy of 17-hydoxyprogesterone caproate therapy in a subject thereof, the method comprising: detecting a concentration of 17α-hydroxyprogesteryl 6-hydroxycaproate in the subject.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A & 1B depict human myometrial tissue contraction parameters (percentage) normalized to control. FIG. 1A depicts contraction frequency; FIG. 1B depicts contraction force (AUC). *p<0.05, **p<0.01, ***p<0.001.

FIGS. 2A-2F depict the effects of inflammatory cytokine production by monocytes treated with one of HPC—OH, HPC, and P4 as analyzed in Example 2. Production of the following cytokines were analyzed: FIG. 2A, IL-1α; FIG. 2B, IL-1β; FIG. 2C, IL-6; FIG. 2D, IL-8; FIG. 2E, IL-10; and FIG. 2F, TNFα.

FIG. 2G depicts the effects of MCP-1 production by monocytes treated with one of HPC—OH, HPC, and P4 as analyzed in Example 2.

FIG. 2H depicts MLC20 phosphorylation after treatment of HPC-primed myometrial cells as analyzed in Example 2.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

As used in this application, including the appended claims, the singular forms “a,” “an,” and “the” include plural references, unless the content clearly dictates otherwise, and are used interchangeably with “at least one” and “one or more.”

As used herein, “preterm delivery” and “preterm birth” are used interchangeably herein to refer to delivery or birth at a gestational age less than 37 completed weeks. Other commonly used subcategories of preterm birth delineate moderately preterm (birth at 33 to 37 weeks of gestation), very preterm (birth at less than 33 weeks of gestation), and extremely preterm (birth at less than 28 weeks of gestation).

The term “sample” as used herein refers to a sample of a body fluid, to a sample of separated cells, or to a sample from a tissue or an organ in a subject. Samples of body fluids can be obtained by well-known techniques and include, but are not limited to, samples of blood, plasma, serum, liquor or urine. In some cases, body fluid samples are obtained by venopuncture, arterial puncture or ventricular puncture. Tissue or organ samples may be obtained from any tissue or organ by, for example, biopsy. Separated cells may be obtained from the body fluids or the tissues or organs by separating techniques such as centrifugation or cell sorting. In some embodiments, cell-, tissue- or organ samples are obtained from those cells, tissues or organs which express or produce the peptides referred to herein.

In accordance with the present disclosure, novel methods for preventing (also referred to herein as reducing the incidence of) preterm delivery in pregnant female subjects are disclosed. In particular, it has now been found that monohydroxylated 17α-hydroxyprogesterone caproate (HPC—OH), and particularly, 17α-hydroxyprogesteryl 6-hydroxycaproate (formula shown below), can be administered to a pregnant female subject to reduce contractility, reduce inflammation, and/or reduced the incidence of preterm delivery.

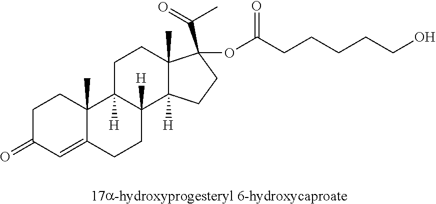

When monohydroxylated 17α-hydroxyprogesterone caproate (HPC—OH) is administered in a unit dose or unit dosage form, it should be understood that the unit dose or unit dosage form can be in a single or divided form as discussed more fully below. Typically, the unit dose or unit dosage form will include a therapeutically effective amount of monohydroxylated 17α-hydroxyprogesterone caproate (HPC—OH) for reducing contractility, reducing inflammation and/or reducing the incidence of preterm delivery in a pregnant female subject. As used herein, “reducing contractility” refers to reducing one or both of contractile force and frequency.

By way of example, in one illustrative embodiment, the unit dose or unit dosage form includes monohydroxylated 17α-hydroxyprogesterone caproate (HPC—OH), and the unit dose or unit dosage includes monohydroxylated 17α-hydroxyprogesterone caproate (HPC—OH) in a therapeutically effective amount of less than 250 mg/week, administered for one to twenty weeks, including from one to two weeks. More generally, the monohydroxylated 17α-hydroxyprogesterone caproate (HPC—OH) can be administered to the subject in amounts ranging from about 100 to about 500 mg/week for a period of from about one to twenty weeks.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the condition, disease or disorder being treated. In one aspect, the therapeutically effective amount is that which may treat or alleviate the condition, disease or symptoms of the disease at a reasonable benefit/risk ratio applicable to any medical treatment. However, it is to be understood that the total weekly usage of the monohydroxylated 17α-hydroxyprogesterone caproate (HPC—OH) described herein may be decided by the attending physician within the scope of sound medical judgment. The specific therapeutically-effective dose level for any particular subject will depend upon a variety of factors, including the severity of the condition being treated; the specific composition employed; the age, body weight, general health, gender and diet of the subject: the time of administration, route of administration, and the duration of the treatment; drugs used in combination or coincidentally with the monohydroxylated 17α-hydroxyprogesterone caproate (HPC—OH); and like factors well known to the researcher, veterinarian, medical doctor or other clinician of ordinary skill.

It is also appreciated that the therapeutically effective amount is advantageously selected with reference to any toxicity, or other undesirable side effect, that might occur during administration of the monohydroxylated 17α-hydroxyprogesterone caproate (HPC—OH).

It is to be understood that in the methods described herein, the monohydroxylated 17α-hydroxyprogesterone caproate (HPC—OH) can be administered by any suitable means. Where the monohydroxylated 17α-hydroxyprogesterone caproate (HPC—OH) is administered in separate dosage forms, the dosages may be administered via the same or different routes of administration.

The term “administering” as used herein includes all means of introducing the monohydroxylated 17α-hydroxyprogesterone caproate (HPC—OH) described herein to the subject, including, but are not limited to, oral (po), intravenous (iv), intramuscular (im), subcutaneous (sc), transdermal, inhalation, buccal, ocular, sublingual, vaginal, rectal, and the like. The monohydroxylated 17α-hydroxyprogesterone caproate (HPC—OH) described herein may be administered in unit dosage forms and/or formulations containing conventional nontoxic pharmaceutically-acceptable carriers, adjuvants, and vehicles. Some examples of materials which can serve as pharmaceutically acceptable carriers, adjuvants, and vehicles include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethyl-polyoxypropyl-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as castor oil, peanut oil, cottonseed oil; safflower oil; sesame oil; olive oil; corn oil and soybean oil; benzyl benzoate; glycols such a propyl glycol or polyethyl glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustratively, administering includes local use, such as when administered locally to a particular organ or tissue system. Illustrative local administration may be performed using parenteral delivery where the monohydroxylated 17α-hydroxyprogesterone caproate (HPC—OH) is deposited locally to the site without general distribution to multiple other non-target sites in the subject being treated. It is further appreciated that local administration may be directly in the effected site, or locally in the surrounding tissue. Similar variations regarding local delivery to particular tissue types, such as organs, and the like, are also described herein. Illustratively, HPC—OH may be administered directly to the vascular system including, but not limited to, intraventricular, intra-arterial, intramuscular, and subcutaneous routes of administration by delivery via needles and/or catheters with or without pump devices.

Depending upon the severity of the condition as described herein, the route of administration and/or whether the monohydroxylated 17α-hydroxyprogesterone caproate (HPC—OH) is administered locally or systemically, a wide range of permissible dosages are contemplated herein, including doses of 250 mg/weekly or less. The dosages may be single or divided, and may be administered according to a wide variety of protocols, e.g., every other day, once a week, once a month, and the like. In each of these cases it is understood that the therapeutically effective amounts described herein correspond to the instance of administration, or alternatively to the total daily, weekly, or month dose, as determined by the dosing protocol.

In making the pharmaceutical compositions of the compounds described herein, a therapeutically effective amount of the monohydroxylated 17α-hydroxyprogesterone caproate (HPC—OH) in any of the various forms described herein may be mixed with one or more excipients, diluted by one or more excipients, or enclosed within such a carrier, which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

The effective use of the monohydroxylated 17α-hydroxyprogesterone caproate (HPC—OH), and methods described herein for reducing the incidence of preterm delivery using the monohydroxylated 17α-hydroxyprogesterone caproate (HPC—OH) may be based upon animal models, such as murine, canine, porcine, and non-human primate animal (e.g., Rhesus macaques) models of disease.

In other embodiments, it has been found herein that monohydroxylated 17α-hydroxyprogesterone caproate (HPC—OH), and particularly, 17α-hydroxyprogesteryl 6-hydroxycaproate, may be used as a diagnostic tool for monitoring the efficacy of 17-hydoxyprogesterone caproate (HPC) treatment. Generally, in these embodiments, methods for monitoring the efficacy of 17-hydoxyprogesterone caproate therapy in a subject includes: detecting a concentration of monohydroxylated 17α-hydroxyprogesterone caproate (HPC—OH) in the subject.

Determining the concentration or level of monohydroxylated 17α-hydroxyprogesterone caproate (HPC—OH) relates to measuring the amount or concentration, including semi-quantitatively or quantitatively. Measuring can be done directly or indirectly. Direct measuring relates to measuring the amount or concentration of the steroid based on a signal which is obtained from the steroid itself and the intensity of which directly correlates with the number of molecules of the steroid present in the sample. Such a signal, sometimes referred to herein as intensity signal, may be obtained by measuring an intensity value of a specific physical or chemical property of the steroid. Indirect measuring includes measuring of a signal obtained from a secondary component (i.e. a component not being the steroid itself) or a biological read out system such as measurable cellular responses, ligands, labels, or enzymatic reaction products.

In accordance with the present disclosure, determining the amount of monohydroxylated 17α-hydroxyprogesterone caproate (HPC—OH) can be achieved by all known means for determining the amount of a steroid in a sample. Said means comprise immunoassay devices and methods which may utilize labeled molecules in various sandwich, competition, or other assay formats. Said assays generally develop a signal which is indicative for the presence or absence of the steroid. Moreover, in some embodiments, the signal strength may be correlated directly or indirectly (e.g. reverse-proportional) to the amount of steroid present in a sample. Further suitable methods include measuring a physical or chemical property specific for the steroid. Said methods comprise biosensors, optical devices coupled to immunoassays, biochips, analytical devices such as mass-spectrometers, NMR-analyzers, or chromatography devices. Further, methods include micro-plate ELISA-based methods, fully-automated or robotic immunoassays, CBA (an enzymatic Cobalt Binding Assay), and latex agglutination assays, homogenous and heterogeneous immune assays, competitive and non-competitive immune assays.

According to various embodiments, determining the amount of a monohydroxylated 17α-hydroxyprogesterone caproate (HPC—OH) comprises the steps of (a) contacting the peptide with a specific ligand, (b) (optionally) removing non-bound ligand, (c) measuring the amount of bound ligand. The bound ligand will generate an intensity signal. Binding according to the present disclosure includes both covalent and non-covalent binding.

A ligand according to the present disclosure can be any compound, e.g., a peptide, polypeptide, nucleic acid, or small molecule, binding to the monohydroxylated 17α-hydroxyprogesterone caproate (HPC—OH) described herein. Exemplary ligands include antibodies, nucleic acids, peptides or polypeptides such as receptors or binding partners for monohydroxylated 17α-hydroxyprogesterone caproate (HPC—OH). Methods of preparing such ligands are well-known in the art. For example, identification and production of suitable antibodies or aptamers is also offered by commercial suppliers. The person skilled in the art is familiar with methods to develop derivatives of such ligands with higher affinity or specificity. For example, random mutations can be introduced into the nucleic acids, peptides or polypeptides. These derivatives can then be tested for binding according to screening procedures known in the art, e.g. phage display.

Means for the detection of monohydroxylated 17α-hydroxyprogesterone caproate (HPC—OH) may include, for example, antibodies to monohydroxylated 17α-hydroxyprogesterone caproate (HPC—OH), including polyclonal and monoclonal antibodies, as well as fragments thereof, such as Fv, Fab and F(ab)2 fragments that are capable of binding monohydroxylated 17α-hydroxyprogesterone caproate (HPC—OH). The means for the detection of monohydroxylated 17α-hydroxyprogesterone caproate (HPC—OH) of the present disclosure also include single chain antibodies, chimeric, humanized hybrid antibodies wherein amino acid sequences of a non-human donor antibody exhibiting a desired antigen-specificity are combined with sequences of a human acceptor antibody. Also included is an anti-monohydroxylated 17α-hydroxyprogesterone caproate (HPC—OH) antibody from a mammalian species, such as an antibody selected from human, rat, mouse, goat, sheep, cattle, and camel.

The ligand may also be “tagged” with one or more tags as known in the art. Such tags may then be targets for higher order ligands. Suitable tags include biotin, digoxygenin, His-Tag, Glutathion-S-Transferase, FLAG, GFP, myc-tag, influenza A virus haemagglutinin (HA), maltose binding protein, and the like. Suitable labels are any labels detectable by an appropriate detection method. Typical labels include gold particles, latex beads, acridan ester, luminol, ruthenium, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. Typical radioactive labels include 35S, 125I, 32P, 33P and the like. A radioactive label can be detected by any method known and appropriate, e.g. a light-sensitive film or a phosphor imager. Suitable measurement methods according the present disclosure also include, for example, precipitation (particularly immunoprecipitation), electrochemiluminescence (electro-generated chemiluminescence), RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry or nephelometry, or solid phase immune tests. Further methods known in the art (such as gel electrophoresis, 2D gel electrophoresis, SDS polyacrylamid gel electrophoresis (SDS-PAGE), Western Blotting, and mass spectrometry), can be used alone or in combination with labeling or other detection methods as described above.

In one particularly suitable embodiment, the method of detecting a concentration of 17α-hydroxyprogesteryl 6-hydroxycaproate includes: contacting, in vitro, a portion of a sample selected from the group consisting of serum, plasma and blood with an antibody having specific binding affinity for 17α-hydroxyprogesteryl 6-hydroxycaproate, thereby forming a complex of the antibody and 17α-hydroxyprogesteryl 6-hydroxycaproate, the antibody having a detectable label; separating the complex formed in said step of contacting from antibody not comprising the complex; quantifying a signal from the detectable label of the antibody comprising the complex formed in said step of contacting, the signal being proportional to an amount of 17α-hydroxyprogesteryl 6-hydroxycaproate in the sample, whereby a concentration of 17α-hydroxyprogesteryl 6-hydroxycaproate in the sample is calculated.

It is believed that when the concentration of 17α-hydroxyprogesteryl 6-hydroxycaproate is less than 6 ng/ml, including less than 5 ng/ml, including less than 4 ng/ml, including less than 3 ng/ml, including less than 2 ng/ml, and even more suitably, including less than 1 ng/ml, the concentration of 17-hydoxyprogesterone caproate (HPC) is not sufficient to prevent preterm birth, and thus, additional HPC is required. Accordingly, under these conditions, the method further includes administering HPC to the subject to raise HPC concentration.

The disclosure will be more fully understood upon consideration of the following non-limiting Examples.

### EXAMPLES

### Example 1

In this Example, the effect of the monohydroxylated 17α-hydroxyprogesterone caproate (HPC—OH), 17α-hydroxyprogesteryl 6-hydroxycaproate, as compared to its parent compound, 17-hydoxyprogesterone caproate (HPC), and progesterone (P4) was analyzed in the setting of oxytocin-induced uterine contractility in human myometrial tissue.

Uterine contractility was studied in human myometrial tissue treated with P4, HPC, and HPC—OH at 10⁻⁶ M. Myometrial biopsies were taken from the superior aspect of a hysterotomy incision at the time of scheduled, unlabored term cesarean deliveries. Myometrial strips (10 mm) were mounted in a digital myograph system (DMT820MS) and maintained in modified Krebs buffer solution at physiologic temperature (37° C.) with constant premixed gas inflow. Oxytocin-induced (100 nm) contractility was measured before and after treatment with HPC—OH, HPC, or P4 (1 μm) for 30 minutes. Myometrial contractile frequency and force (AUC) were determined for each treatment over a 4-hour period and normalized to a matched vehicle control (DMSO). Statistical analysis was by student t-test (GraphPad Prism 7.0a). Statistical significance was defined as p<0.05.

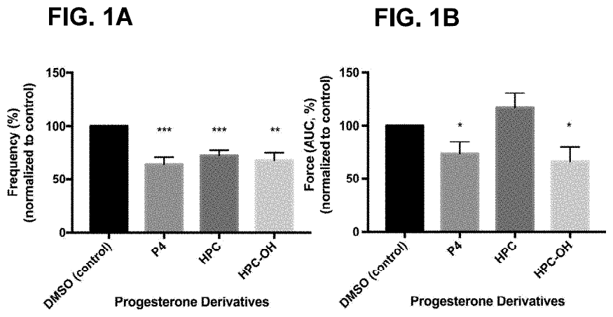

Myometrial biopsies were taken from 13 unlabored cesarean deliveries. As shown in FIG. 1A, as compared to an untreated control, oxytocin-induced contractile frequency was decreased by progesterone (63%+/−18%, p<0.0001), HPC (71%+/−15%, p<0.0001), and HPC—OH (67%+/−21%, p=0.0001). Further, only P4 and HPC—OH decreased oxytocin-induced contractile force (75%+/−30%, p=0.033 and 66%+/−33%, p=0.008, respectively) compared to the control (FIG. 1B). HPC—OH demonstrated a greater reduction in contractile force when compared directly to HPC (64%+/−33%, p=0.028) (FIG. 1B).

Based on the foregoing, HPC—OH, a derivative produced from the metabolism of HPC, demonstrated greater reduction of myometrial contractility than its parent compound.

### Example 2

In this Example, the ability of HPC—OH to decrease systemic inflammation-induced myometrial contractility was compared to HPC and progesterone (P4).

An in vitro model of systemic inflammation-induced uterine contractility was created with mononuclear and myometrial cell cultures to assess the effect of progestogen treatment. THP-1 mononuclear cells were cultured with HPC—OH (17α-hydroxyprogesteryl 6-hydroxycaproate), HPC, or P4 (all at 100 nM) for 24 hours then treated with lipopolysaccharide (LPS, 1 μg/mL) for 24 hours. Culture supernatant was characterized by multiplex assay for 7 inflammation-related cytokines: IL-1α, IL-10, IL-6, IL-8, IL-10, TNFα, and MCP-1. Next, PHM1-41 human pregnancy myometrial cells pretreated with HPC for 24 hours were incubated with THP-1 culture supernatant (from the previous step) for 24 hours. Myometrial contractility was assessed after stimulation with 100 μM oxytocin by in-cell western (ICW) quantification of myosin light chain (MLC20) phosphorylation, a molecular event preceding contractility.

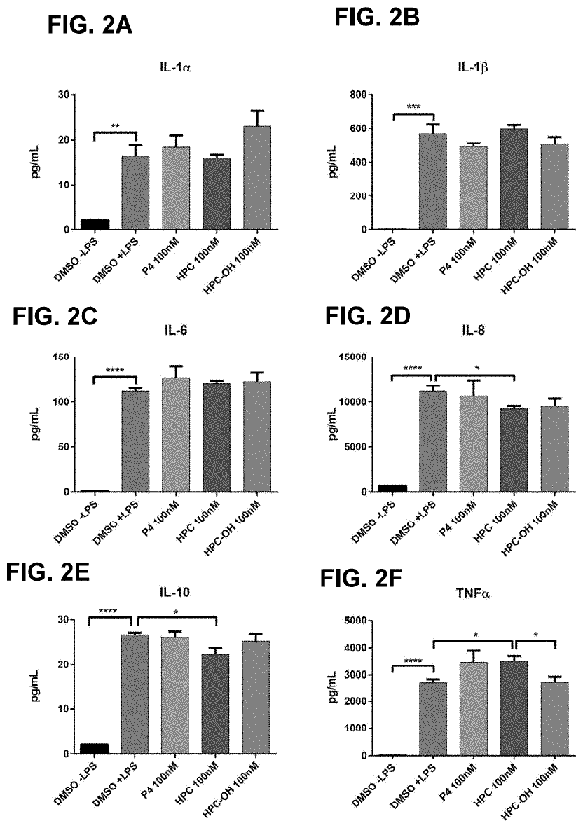

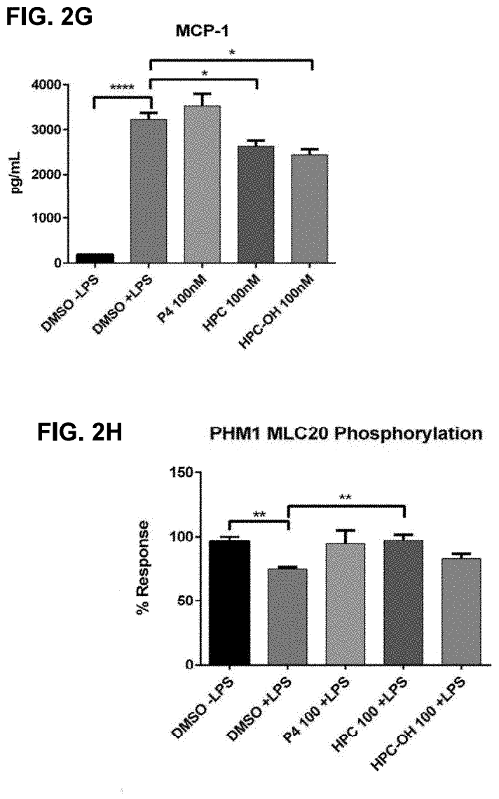

As shown in FIGS. 2A-2F, treatment of monocytes with LPS increased inflammatory cytokine production compared to unstimulated controls (p<0.01). At 100 nM, HPC—OH treatment did not result in a statistically significant change in monocyte production of IL-1α, IL-1β, IL-6, or IL-8 (FIGS. 2A-2D). Treatment with HPC—OH (p=0.45) or P4 (p=0.70) did not diminish production of IL-10, an anti-inflammatory cytokine (FIG. 2E). In contrast, HPC treatment significantly reduced IL-10 production (p=0.05) (FIG. 2E). HPC (p=0.02), but not HPC—OH (p=0.95), treatment was seen to significantly increase TNFα production (FIG. 2F). Levels of TNFα produced after exposure to HPC—OH were significantly lower than those produced after exposure to HPC (p=0.046) (FIG. 2F). As shown in FIG. 2G, production of MCP-1, a chemokine attractant for immune cells, was decreased in the presence of HPC (p=0.03) and HPC—OH (p=0.01). MLC20 phosphorylation assessed by ICW demonstrated a decrease after treatment with LPS-treated culture supernatant compared to untreated supernatant (p=0.005). Increased phosphorylation was noted after exposure to P4 (p=0.008). MLC20 phosphorylation after treatment of HPC-primed myometrial cells with HPC—OH treated supernatant trended towards a significant decline from HPC treated supernatant (p=0.06) (FIG. 2H).

Based on the foregoing, HPC and P4 mitigate LPS-induced systemic inflammation and myometrial contractility in a dose dependent manner HPC inhibits a broader range of inflammatory cytokines, resulting in reduced contractility. This suggests that HPC may have efficacy in reducing spontaneous preterm birth triggered by systemic infection.

### Example 3

In this Example, the pharmacokinetics of various monohydroxylated 17α-hydroxyprogesterone caproates (HPC—OH) were analyzed and compared to the pharmacokinetics of HPC.

Female patients with a singleton pregnancy received intramuscular doses of 250 mg hydroxyprogesterone caproate for the reduction of preterm birth starting between 16 weeks 0 days and 20 weeks 6 days. More particularly, the patients were divided into three sample groups: Group 1 (patients at 16-20 weeks gestation); Group 2 (patients at 24-28 weeks gestation); and Group 3 (patients at 32-36 weeks gestation). All patients had blood drawn daily for 7 days to evaluate pharmacokinetics. Pharmacokinetics were analyzed according to the assay method as described in Zhang et al., Journal of Pharmaceutical and Biomedical Analysis 48 (20088): 1174-1180, which is incorporated herein by reference to the extent it is consistent herewith.

A summary of the mean (standard deviation) PK parameters for the HPC—OH is shown in Table 1.

TABLE 1
Summary of Mean (Standard Deviation) PK Parameters
for Hydroxyprogestrone Caproate

| | Cₘₐₓ | Tₘₐₓ | AUC₍₁₋ₜ₎^{b} |
|---|---|---|---|
| Group (N) | (ng/mL) | (days)^{a} | (ng · hr/mL) |
| Group 1 (N = 6) | 5.0 (1.5) | 5.5 (2.0-7.0) | 571.4 (195.2) |
| Group 2 (N = 8) | 12.5 (3.9) | 1.0 (0.9-1.9) | 1269.6 (285.0) |
| Group 3 (N = 11) | 12.3 (4.9) | 2.0 (1.0-3.0) | 1268.0 (511.6) |

For all three groups, peak concentration (Cₘₐₓ) and area under the curve (AUC_{(1-7 days)}) of the mono-hydroxylated metabolites were approximately 3-7-fold lower than the respective parameters for the parent drug, hydroxyprogesterone caproate. While di-hydroxylated and tri-hydroxylated metabolites were also detected in human plasma to a lesser extent, no meaningful quantitative results could be derived due to the absence of reference standards for these multiple hydroxylated metabolites. The relative activity and significance of these metabolites are not known.

The elimination half-life of hydroxyprogesterone caproate, as evaluated from 4 patients in this Example who reached full-term in their pregnancies, was 16.4 (±3.6) days. The elimination half-life of the mono-hydroxylated metabolites was 19.7 (±6.2) days.

What is claimed is:

1. A method of reducing the incidence of preterm delivery in a pregnant female subject, the method comprising administering 17α-hydroxyprogesteryl 6-hydroxycaproate to the subject.

2. The method as set forth in claim 1 wherein the pregnant female subject is less than 37 weeks gestation.

3. (canceled)

4. The method as set forth in claim 1 comprising administering less than 250 mg/weekly 17α-hydroxyprogesteryl 6-hydroxycaproate to the subject.

5. The method as set forth in claim 1 wherein the 17α-hydroxyprogesteryl 6-hydroxycaproate is administered by an administration method selected from the group consisting of intravenously, intraarterially, intraperitoneally, epidurially, intraurethrally, intrasternally, and intramuscularly.

6. The method as set forth in claim 1 wherein the 17α-hydroxyprogesteryl 6-hydroxycaproate is administered intravenously using an oil-based pharmaceutically-acceptable vehicle.

7. A method of reducing contractility in a pregnant female subject, the method comprising administering 17α-hydroxyprogesteryl 6-hydroxycaproate to the subject.

8. The method as set forth in claim 7 wherein the pregnant female subject is less than 37 weeks gestation.

9. (canceled)

10. The method as set forth in claim 7 comprising administering less than 250 mg/weekly 17α-hydroxyprogesteryl 6-hydroxycaproate to the subject.

11. The method as set forth in claim 7 wherein the 17α-hydroxyprogesteryl 6-hydroxycaproate is administered by an administration method selected from the group consisting of intravenously, intraarterially, intraperitoneally, epidurially, subcutaneously, intraurethrally, intrasternally, and intramuscularly.

12. The method as set forth in claim 7 wherein the 17α-hydroxyprogesteryl 6-hydroxycaproate is administered intravenously using an oil-based pharmaceutically-acceptable vehicle.

13. (canceled)

14. The method of claim 7, wherein contractile force is reduced after administration of 17α-hydroxyprogesteryl 6-hydroxycaproate to the subject.

15. The method of claim 7, wherein contractile frequency is reduced after administration of 17α-hydroxyprogesteryl 6-hydroxycaproate to the subject.

16. A method of reducing inflammation in a pregnant female subject, the method comprising administering 17α-hydroxyprogesteryl 6-hydroxycaproate to the subject.

17. The method as set forth in claim 16 wherein the pregnant female subject is less than 37 weeks gestation.

18. (canceled)

19. The method as set forth in claim 16 comprising administering less than 250 mg/weekly 17α-hydroxyprogesteryl 6-hydroxycaproate to the subject.

20. The method as set forth in claim 16 wherein the 17α-hydroxyprogesteryl 6-hydroxycaproate is administered by an administration method selected from the group consisting of intravenously, intraarterially, intraperitoneally, epidurially, intraurethrally, intrasternally, and intramuscularly.

21. The method as set forth in claim 16 wherein the 17α-hydroxyprogesteryl 6-hydroxycaproate is administered intravenously using an oil-based pharmaceutically-acceptable vehicle.

22. A method of monitoring the efficacy of 17-hydoxyprogesterone caproate therapy in a subject thereof, the method comprising: detecting a concentration of 17α-hydroxyprogesteryl 6-hydroxycaproate in the subject.

23. The method as set forth in claim 22, wherein the method of detecting a concentration of 17α-hydroxyprogesteryl 6-hydroxycaproate comprises:
contacting, in vitro, a portion of a sample selected from the group consisting of serum, plasma and blood with an antibody having specific binding affinity for 17α-hydroxyprogesteryl 6-hydroxycaproate, thereby forming a complex of the antibody and 17α-hydroxyprogesteryl 6-hydroxycaproate, the antibody having a detectable label;
separating the complex formed in said step of contacting from antibody not comprising the complex;
quantifying a signal from the detectable label of the antibody comprising the complex formed in said step of contacting, the signal being proportional to an amount of 17α-hydroxyprogesteryl 6-hydroxycaproate in the sample, whereby a concentration of 17α-hydroxyprogesteryl 6-hydroxycaproate in the sample is calculated.

24. The method as set forth in claim 22, wherein if the concentration of 17α-hydroxyprogesteryl 6-hydroxycaproate in the subject is less than 6 ng/ml, the method further comprises administering 17-hydroxyprogesterone caproate to the subject.

25. (canceled)
